(19) United States
(12) Patent Application Publication
(10) Pub. No.: US 2026/0102587 A1
(43) Pub. Date: Apr. 16, 2026

(54) APPARATUS AND METHOD FOR POSITIONING A MEDICAL IMAGING DEVICE

(71) Applicant: Kevin Randall Hayes, Texarkana, TX (US)

(72) Inventor: Kevin Randall Hayes, Texarkana, TX (US)

(21) Appl. No.: 19/232,696

(22) Filed: Jun. 9, 2025

Related U.S. Application Data
(60) Provisional application No. 63/658,356, filed on Jun. 10, 2024.

(51) Int. Cl. A61M 25/01; A61M 25/02

(52) U.S. Cl. CPC A61M 25/0127; A61M 2025/0175; A61M 2025/024

(57) ABSTRACT

An apparatus and method for positioning a medical imaging device during a medical procedure. The apparatus may include a positioning portion that positions the medical imaging device during the medical procedure. The positioning portion may include a base and at least one member coupled to the base. The at least one member may grasp at least a portion of the medical imaging device and substantially rotationally fix the medical imaging device about a rotational axis. The apparatus may further include a support portion coupled to the positioning portion that supports the positioning portion.

### CROSS-REFERENCE TO RELATED PATENT APPLICATION

This patent application claims the benefit of and priority to U.S. Provisional Patent Application No. 63/658,356 , filed Jun. 10, 2024, which is incorporated herein by reference in its entirety and for all purposes.

### TECHNICAL FIELD

The present application relates generally to an apparatus and method for positioning a medical imaging device or a plurality of medical imaging devices.

### BACKGROUND

In some medical procedures (e.g., intracardiac echocardiography, catheter ablation for an atrial fibrillation, a ventricular tachycardia, or a supraventricular tachycardias (SVT), implementation of cardiac defibrillations (ICDs), cardiac pacemakers, or loop recorders for arrhythmia detection, etc.), a medical imaging device (e.g., a catheter, a coronary sinus (CS) catheter, an echo-cardiac catheter, etc.) is utilized to provide images or videos of a human organ (e.g., a heart, etc.). The medical imaging device may be rotated and shifted to provide various perspectives and/or angles of the human organ. Conventionally, the medical imaging device is maintained at a particular rotational angle and/or position using an object that is placed above the medical imaging device, such as a wet towel. However, the wet towel may be difficult to control to maintain a desired rotation and/or position of the medical imaging device. Additionally, the wet towel can come in contact with a patient on which the medical procedure is being performed, thereby affecting a temperature of the patient (i.e., the wet towel may have a lower temperature than a temperature of the patient, thus decreasing the temperature of the patient, etc.). Furthermore, the wet towel may be a breeding ground for bacteria.

### SUMMARY

Embodiments disclosed herein provide an apparatus for positioning the medical imaging device that alleviates the need for using objects, such as the wet towel, to position the medical imaging device.

In one embodiment, an apparatus for positioning a medical imaging device during a medical procedure includes a positioning portion that positions the medical imaging device during the medical procedure. The positioning portion includes a base and at least one member coupled to the base and being bendable. The at least one member grasps at least a portion of the medical imaging device and substantially rotationally fixes the medical imaging device about a rotational axis. The apparatus further includes a support portion coupled to the positioning portion and that supports the positioning portion.

In some embodiments, the support portion includes a telescopic arm.

In some embodiments, the support portion includes an adjustable arm. The adjustable arm includes a first member and a second member rotatably coupled to the first member.

In some embodiments, the first member and the second member are substantially rigid.

In some embodiments, the support portion includes a first end proximate the positioning portion a second end opposite the first end. The apparatus further includes a coupling device proximate the second end. The coupling device couples to an object and affixes at least a portion of the support portion to the object.

In some embodiments, the coupling device is at least one of a magnet, a clamp, or a screw.

In some embodiments, the coupling device is couplable along a length of the object.

In some embodiments, the at least one member includes three members.

In some embodiments, the at least one member includes at least one grippable region that contacts the medical imaging device and prevents substantial rotation about the rotational axis.

In some embodiments, the at least one member is manually disengageable to manually rotate the medical imaging device about the rotational axis.

In some embodiments, the support portion includes a telescopic arm proximate a first end of the support portion. The telescopic arm is rotatably coupled to the base of the positioning portion. The telescopic arm includes a first telescopic end proximate the base of the positioning portion and a second telescopic end opposite of the first telescopic end. The support portion further includes an adjustable arm coupled to the telescopic arm proximate the second telescopic end of the telescopic arm. The adjustable arm includes a first member and a second member rotatably coupled to the first member. The support portion further includes a clamp coupled to the first member of the adjustable arm. The clamp couples to an object and spatially fixes the support portion to the object.

In some embodiments, the apparatus includes a weight coupled to the support portion. The positioning portion defines a plurality of slots. Each of the plurality of slots selectively receives the support portion.

In another embodiment, an apparatus for positioning a plurality of medical imaging devices includes a positioning portion that positions the medical imaging device during a medical procedure. The positioning portion includes a clamping body that couples to the medical imaging device. The clamping body includes an inner portion that contacts the medical imaging device and an outer portion opposite of the inner portion. The outer portion includes at least one clamping magnet. The positioning portion further includes a receiving member that couples to the clamping body. The receiving member includes at least one receiving magnet that magnetically couples to the at least one clamping magnet of the clamping body. The apparatus further includes a support portion coupled to the positioning portion and that supports the positioning portion.

In some embodiments, the inner portion of the clamping body includes a plurality of teeth. The plurality of teeth include a plurality of bottom lands. At least one of the plurality of bottom lands is disposed between adjacent teeth of the plurality of teeth. The clamping body further includes a first end, a second end opposite of the first end, and an aperture proximate the first end. The aperture receives a bottom land of the plurality of bottom lands of the plurality of teeth.

In some embodiments, at least one tooth of the plurality of teeth includes rubber.

In some embodiments, the at least one clamping magnet of the clamping body of the positioning portion includes a first clamping magnet and a second clamping magnet adjacent to the first clamping magnet. The at least one receiving magnet of the receiving member of the positioning portion includes a first receiving magnet and a second receiving magnet. The first receiving magnet magnetically couples to the first clamping magnet and the second receiving magnet magnetically couples to the second clamping magnet.

In some embodiments, the receiving member of the positioning portion includes a first arm coupled to the first receiving magnet and a second arm coupled to the second receiving magnet.

In some embodiments, the clamping body is a first clamping body. The at least one clamping magnet of the first clamping body includes a first clamping magnet. The positioning portion further includes a second clamping body that couples to the medical imaging device. The second clamping body includes a second clamping magnet. The receiving member includes a first arm and a second arm. The at least one receiving magnet of the receiving member of the positioning portion includes a first receiving magnet coupled to the first arm and a second receiving magnet coupled to the second arm. The first receiving magnet magnetically couples to the first clamping magnet of the first clamping body. The second receiving magnet magnetically couples to the second clamping magnet of the second clamping body.

In some embodiments, at least one of the first arm or the second arm is telescopic.

In some embodiments, a system includes the apparatus. The at least one clamping magnet and the at least one receiving magnet are electromagnets. The system further includes a switch operatable between a first position and a second position. The system further includes a controller communicatively coupled to the switch, the at least one clamping magnet, and the at least one receiving magnet. The controller, when the switch is in the first position, provides electrical current to at least one of the at least one clamping magnet or the at least one receiving magnet to generate magnetic fields, and, when the switch is in the second position, does not provide the electrical current to the at least one of the at least one clamping magnet or the at least one receiving magnet to prevent or minimize generation of the magnetic fields.

In some embodiments, the switch is a foot pedal.

In yet another embodiment, an apparatus for positioning a plurality of medical imaging devices includes a positioning portion that positions at least one of the plurality of medical imaging devices during a medical procedure. The positioning portion includes a base. The base includes an upper portion and a lower portion opposite of the upper portion. The positioning portion further includes a plurality of clips coupled to the base. At least one of the plurality of clips is biasedly closed and receives a respective medical imaging device of the plurality of medical imaging devices, grasps at least a portion of the respective medical imaging device of the plurality of medical imaging devices, and substantially spatially fixes about a rotational axis the respective medical imaging device of the plurality of medical imaging devices. The apparatus further includes a support portion coupled to the positioning portion and that supports the positioning portion.

In some embodiments, the plurality of clips are coupled to the upper portion of the base of the positioning portion.

In some embodiments, the plurality of clips are coupled to the lower portion of the base of the positioning portion.

In some embodiments, at least one of the plurality of clips includes a C-clip.

In yet another embodiment, an apparatus for positioning a medical imaging device the includes a positioning portion that positions the medical imaging device during a medical procedure. The positioning portion includes a clamping body that couples to the medical imaging device. The clamping body includes an inner portion that contacts the medical imaging device and an outer portion opposite of the inner portion. The outer portion includes a plurality of clamping teeth. The plurality of clamping teeth include a plurality of clamping bottom lands. At least one of the plurality of clamping bottom lands is disposed between adjacent teeth of the plurality of clamping teeth. At least one of the plurality of clamping teeth includes a clamping top land. The positioning portion further includes a receiving housing that couples to the clamping body. The receiving housing includes a clockwise locking tooth that interfaces with at least one of the plurality of clamping bottom lands of the plurality of clamping teeth to prevent rotation of the clamping body in a clockwise direction. The receiving housing further includes a counterclockwise locking tooth that interfaces with at least one of the plurality of clamping bottom lands of the plurality of clamping teeth to prevent rotation of the clamping body in a counterclockwise direction. The apparatus further includes a support portion coupled to the positioning portion and that supports the positioning portion.

In some embodiments, the receiving housing is operable between a first position in which neither of the clockwise locking tooth nor the counterclockwise locking tooth are engaged with the clamping body, such that the clamping body is free to rotate in the clockwise direction and the counterclockwise direction, a second position in which the clockwise locking tooth is engaged with the clamping body and the counterclockwise locking tooth is not engaged with the clamping body, such that the clamping body is prevented from rotating in the clockwise direction and free to rotate in the counterclockwise direction, a third position in which the counterclockwise locking tooth is engaged with the clamping body and the clockwise locking tooth is not engaged with the clamping body, such that the clamping body is prevented from rotating in the counterclockwise direction and free to rotate in the clockwise direction, and a fourth position in which both of the clockwise locking tooth and the counterclockwise locking tooth are engaged with the clamping body, such that the clamping body is prevented from rotating in both the clockwise direction and the counterclockwise direction.

In some aspects, the techniques described herein relate to a system including: a positioning portion configured to position a medical imaging device during a medical procedure, the positioning portion including: a clamping body configured to couple to the medical imaging device, the clamping body including: an inner portion configured to contact the medical imaging device including a plurality of teeth, the plurality of teeth including a plurality of bottom lands, at least one of the plurality of bottom lands disposed between adjacent teeth of the plurality of teeth, and an outer portion opposite of the inner portion, the outer portion including at least one clamping magnet; a first end, a second end opposite of the first end, and an aperture proximate the first end, the aperture configured to receive a bottom land of the plurality of bottom lands of the plurality of teeth; and a receiving member configured to couple to the clamping body, the receiving member including at least one receiving magnet configured to magnetically couple to the at least one clamping magnet of the clamping body; and a support portion coupled to the positioning portion and configured to support the positioning portion, wherein the at least one clamping magnet and the at least one receiving magnet are electromagnets; a switch operable between a first position and a second position; and a controller communicatively coupled to the switch, the at least one clamping magnet, and the at least one receiving magnet, the controller configured to: when the switch is in the first position, provide electrical current to at least one of the at least one clamping magnet or the at least one receiving magnet to generate magnetic fields, and when the switch is in the second position, not provide the electrical current to the at least one of the at least one clamping magnet or the at least one receiving magnet to prevent or minimize generation of the magnetic fields.

In some embodiments, a method for positioning a medical imaging device is described. The method may comprise: positioning the medical imaging device proximate a positioning portion of an apparatus; coupling the medical imaging device at least in part to the positioning portion of the apparatus; and adjusting a rotational position of the apparatus. The method for positioning the medical imaging device, wherein adjusting the rotational position of the apparatus comprises: removing the medical imaging device from the positioning portion of the apparatus; rotating the medical imaging device; and recoupling the medical imaging device to the positioning portion of the medical imaging device. The method of positioning the medical imaging device, wherein adjusting the rotational position of the apparatus comprises: applying a force to the positioning portion of the apparatus to overcome a biasing force of the positioning portion of the apparatus to disengage a support portion of the apparatus from a slot; rotating the positioning portion while the medical imaging device is coupled thereto; and releasing the force to the positioning portion to reapply the biasing force to the positioning portion of the apparatus to engage the support portion with the slot. The method for positioning the medical imaging device, wherein adjusting the rotational position of the apparatus comprises: rotating the positioning portion while the medical imaging device is coupled thereto; applying a force to the positioning portion of the apparatus to overcome a biasing force of the positioning portion of the apparatus to disengage a support portion of the apparatus from a slot; rotating a base of the apparatus to rest upon a support surface; and releasing the force to the positioning portion to reapply the biasing force to the positioning portion.

### BRIEF DESCRIPTION OF THE DRAWINGS

The details of one or more implementations are set forth in the accompanying drawings and the description below. Other features, aspects, and advantages of the disclosure will become apparent from the description, the drawings, and the claims, in which:

FIG. 1A is a side view of a first apparatus for positioning a medical imaging device that includes a first positioning portion, according to an example embodiment;

FIG. 1B is a side view of the first positioning portion of FIG. 1A, according to an example embodiment;

FIG. 1C is a perspective view of the first positioning portion gripping the medical imaging device, according to an example embodiment;

FIG. 2A is a side view of a second apparatus for positioning the medical imaging device that includes a clamping body and a receiving member, according to an example embodiment;

FIG. 2B is a side view of the clamping body, according to an example embodiment;

FIG. 2C is a top view of the clamping body of FIG. 2B, according to an example embodiment;

FIG. 2D is a bottom view of the clamping body of FIGS. 2B and 2C, according to an example embodiment;

FIG. 2E is another side view of the clamping body of FIGS. 2B-2D, according to an example embodiment;

FIG. 3A is perspective view of the medical imaging device and the clamping body, according to an example embodiment;

FIG. 3B is a side view of the receiving member, according to an example embodiment;

FIG. 4A is a side view of another receiving member, according to an example embodiment;

FIG. 4B is a side view of the receiving member of FIG. 4A and two of the clamping bodies, according to an example embodiment;

FIG. 4C is a perspective view of the medical imaging device and the two of the clamping bodies, according to an example embodiment;

FIG. 5A is a side view of the receiving member of FIG. 4A and another clamping body, according to an example embodiment;

FIG. 5B is a perspective view of the medical imaging device and the clamping body of FIG. 5A, according to an example embodiment;

FIGS. 6A-6D are side views of a third apparatus for positioning medical imaging devices, according to various example embodiments;

FIG. 7A-7C are perspective views of the medical imaging device and clips of the third apparatus, according to various example embodiments;

FIG. 8A is a side view of a fourth apparatus for positioning the medical imaging device, according to an example embodiment;

FIG. 8B is a side view of a clamping body of the fourth apparatus, according to an example embodiment;

FIG. 8C is another side view of the clamping body of FIG. 8B, according to an example embodiment;

FIGS. 8D-8G are side views of a positioning portion of the fourth apparatus, according to various example embodiments;

FIG. 9A is a top view of a fifth apparatus for positioning the medical imaging device, according to an example embodiment;

FIG. 9B is a cross-sectional view of the fifth apparatus along line 9B-9B in FIG. 9A, according to an example embodiment;

FIG. 9C is a side view of the fifth apparatus, according to another example embodiment; and FIG. 9D is a side view of the fifth apparatus, according to another example embodiment.

It will be recognized that the Figures are schematic representations for purposes of illustration. The Figures are provided for the purpose of illustrating one or more implementations with the explicit understanding that the Figures will not be used to limit the scope or the meaning of the claims.

### DETAILED DESCRIPTION

Following below are more detailed descriptions of various concepts related to, and implementations of, methods, apparatuses, and systems for positioning a medical imaging device. The various concepts introduced above and discussed in greater detail below may be implemented in any of a number of ways, as the described concepts are not limited to any particular manner of implementation. Examples of specific implementations and applications are provided primarily for illustrative purposes.

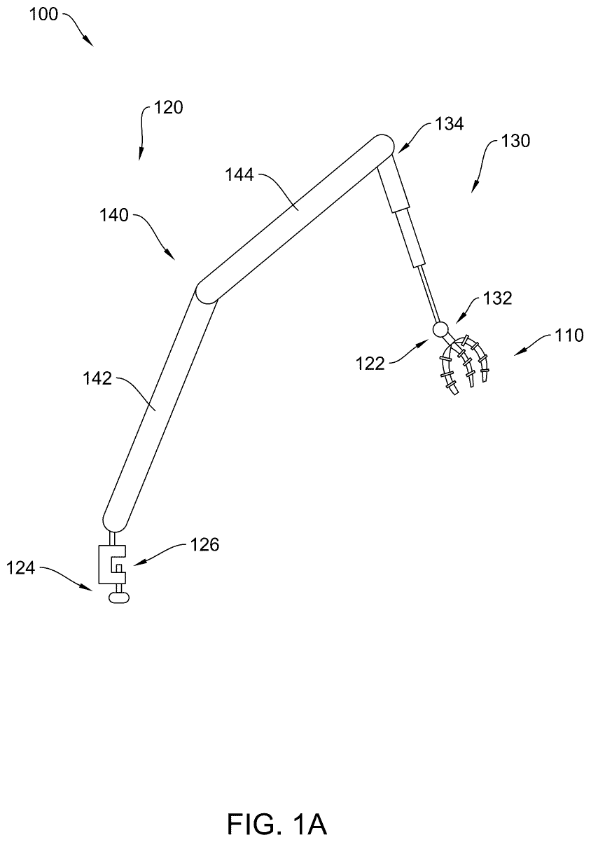

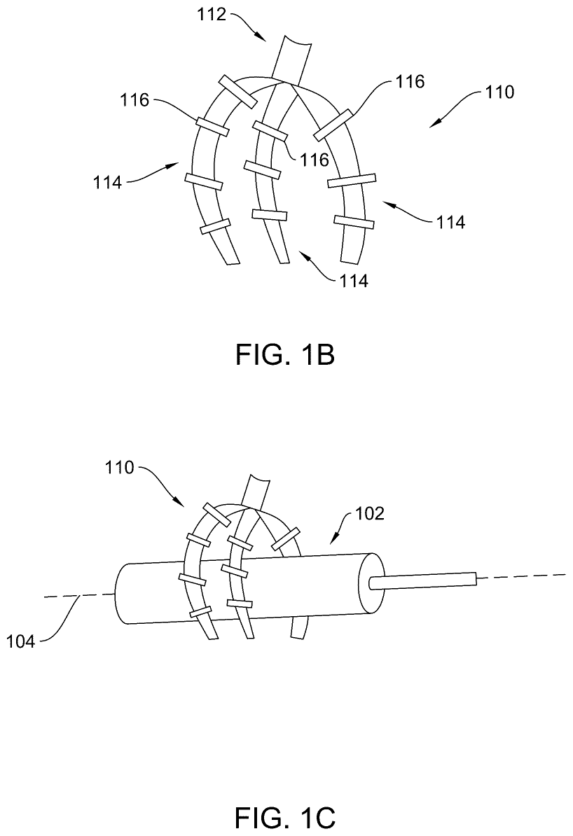

FIG. 1A illustrates a first apparatus 100 (e.g., an apparatus, etc.) for positioning a medical imaging device 102 during a medical procedure, according to an example embodiment. The medical imaging device 102 can include a catheter, a coronary sinus (CS) catheter, an echo-cardiac catheter, and/or the like. The medical procedure can include an intracardiac echocardiography, a catheter ablation for an atrial fibrillation, a ventricular tachycardia, or a supraventricular tachycardias (SVT), implementation of cardiac defibrillations (ICDs), cardiac peacemakers, or loop recorders for arrhythmia detection, or the like.

While the medical imaging device 102 is described herein for illustrative purposes, and for ease of description, it should be understood that the first apparatus 100 may be used to positionally and/or rotatably fix other medical devices such as non-imaging catheters.

The first apparatus 100 includes a positioning portion 110. The positioning portion 110 is configured to position the medical imaging device 102 during the medical procedure. As illustrated in FIGS. 1B and 1C, the positioning portion 110 includes a base 112 and at least one member 114 coupled to the base 112. The at least one member 114 may be bendable (e.g., flexible, semi-flexible, etc.). The at least one member 114 is configured to grasp at least a portion of the medical imaging device 102 and substantially rotationally fix the medical imaging device 102 about a rotational axis 104. The at least one member 114 may be manually disengageable to manually rotate the medical imaging device 102 about the rotational axis 104 and manually engageable to manually fix the medical imaging device 102 about the rotational axis 104. In some embodiments, the medical imaging device 102 extends, or substantially extends, along the rotational axis 104.

In some embodiments, the positioning portion 110 includes three of the member 114 (i.e., the at least one member 114 includes three members 114, etc.). In other embodiments, the positioning portion 110 includes less than three (e.g., two, one, etc.) or greater than three (e.g., four, seven, etc.) of the member 114.

The at least one member 114 may include at least one grippable region 116 (e.g., a sticky region, a rubber area, etc.). The at least one grippable region 116 is configured to contact the medical imaging device 102 and prevent the medical imaging device 102 from substantially rotating about the rotational axis 104. In some embodiments, the at least one member 114 includes three of the at least one grippable region 116 (i.e., the at least one grippable region 116 includes three grippable regions 116, etc.). In other embodiments, the at least one member 114 includes less than three (e.g., two, one, etc.) or greater than three (e.g., four, seven, etc.) of the grippable region 116.

As illustrated in FIG. 1A, the first apparatus 100 further includes a support portion 120. The support portion 120 is coupled to the positioning portion 110. The support portion 120 is configured to support the positioning portion 110. The support portion 120 may include a first end 122 proximate the positioning portion 110 and a second end 124 opposite the first end 122. The support portion 120 may include a coupling device 126 (e.g., a fastening device, a fastener, a clamp, a clamping device, etc.) proximate the second end 124. The coupling device 126 is configured to couple to an object (e.g., an operation surface, a surgery table, a patient support surface, a medical device, etc.) and affix at least a portion of the support portion 120 to the object. The coupling device 126 may be at least one of a magnet, a clamp, or a screw. The coupling device 126 may be couplable along a length of the object.

The support portion 120 may include a telescopic arm 130 (e.g., an extendable arm, a retractable arm, etc.) proximate the first end 122 of the support portion 120. The telescopic arm 130 may be rotatably coupled to the base 112 of the positioning portion 110. The telescopic arm 130 may include a first telescopic end 132 proximate the base 112 of the positioning portion 110. The telescopic arm 130 may further include a second telescopic end 134 opposite of the first telescopic end 132.

The support portion 120 may include an adjustable arm 140. The adjustable arm 140 includes a first member 142 and a second member 144 that is rotatably coupled to the first member 142. least one of the first member 142 or the second member 144 may be substantially rigid. The adjustable arm 140 may include additional members to the first member 142 and the second member 144, such as a third member, a fourth member, etc.

The adjustable arm 140 (e.g., the second member 144, etc.) may be coupled to the telescopic arm 130 proximate the second telescopic end 134. The coupling device 126 may be coupled to the first member 142 of the adjustable arm 140.

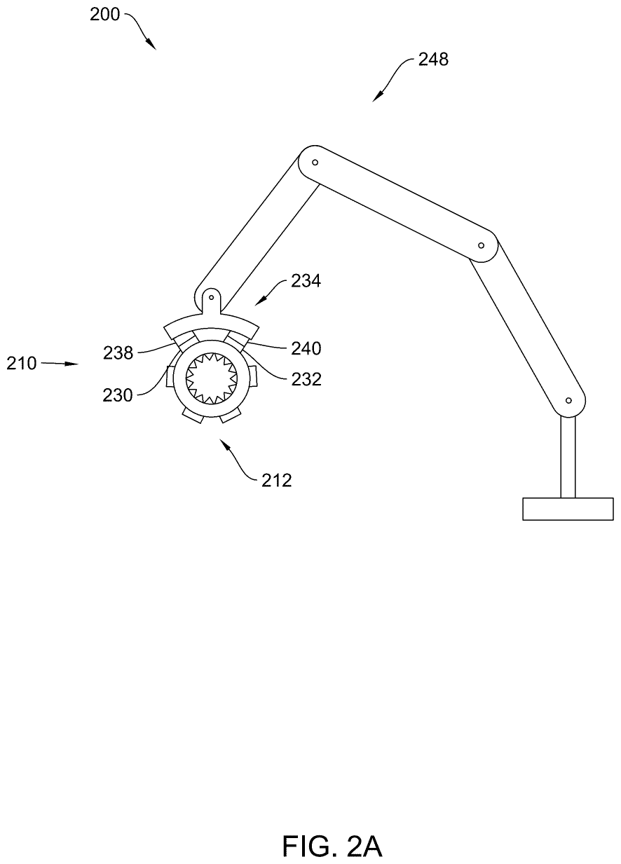

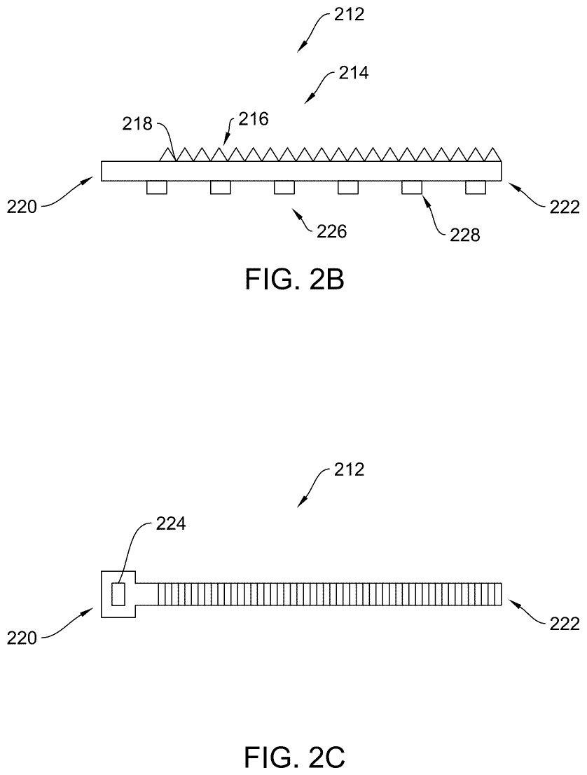

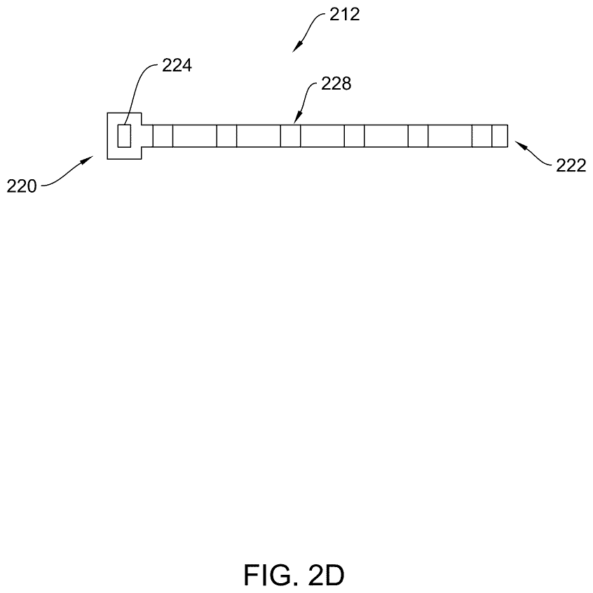

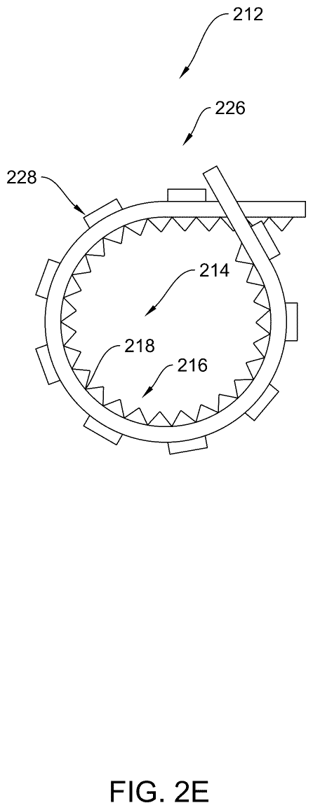

FIG. 2A illustrates a second apparatus 200 (e.g., an apparatus, etc.) for positioning the medical imaging device 102 during the medical procedure, according to an example embodiment. The second apparatus 200 includes a positioning portion 210. The positioning portion 210 is configured to position the medical imaging device 102 during the medical procedure.

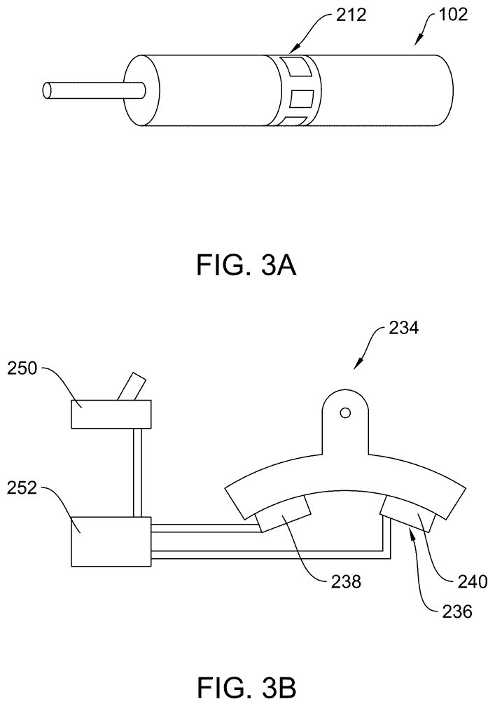

As illustrated in FIGS. 2B and 3A, the positioning portion 210 includes a clamping body 212 (e.g., a clamping extension, a clamping belt, etc.). The clamping body 212 is configured to couple to the medical imaging device 102. The clamping body 212 includes an inner portion 214. The inner portion 214 is configured to contact the medical imaging device 102. The inner portion 214 of the clamping body 212 includes a plurality of teeth 216. At least one of the plurality of teeth 216 is configured to interface with the medical imaging device 102 to prevent slippage between the inner portion 214 of the clamping body 212 and the medical imaging device 102. In some embodiments, at least one tooth of the plurality of teeth 216 is elastically deformable, such that the at least one tooth is configured to at least partially elastically deform when interfacing with the medical imaging device 102, thereby increasing engagement between the at least one tooth and the medical imaging device 102. In some embodiments, the at least one tooth of the plurality of teeth 216 includes rubber. The plurality of teeth 216 includes a plurality of bottom lands 218. At least one of the plurality of bottom lands 218 is disposed between adjacent teeth of the plurality of teeth 216.

The clamping body 212 includes a first end 220 and a second end 222 opposite of the first end 220. The clamping body 212 may include an aperture 224 at or proximate the first end 220. As illustrated in FIG. 2E, the clamping body 212 may be secured to the medical imaging device 102 by wrapping the clamping body 212 around the medical imaging device 102 and tightening the clamping body 212 around the medical imaging device 102 by passing and pulling the second end 222 of the clamping body 212 through the aperture 224. The aperture 224 may be configured to receive a bottom land of the plurality of bottom lands 218 of the plurality of teeth 216 and interface with the bottom land, such that the clamping body 212 is secured to the medical imaging device 102. While a single aperture 224 is shown in FIG. 2C, it should be understood that any number of single apertures 224 may be positioned along the clamping body 212 for tightening of the clamping body 212 in various configurations.

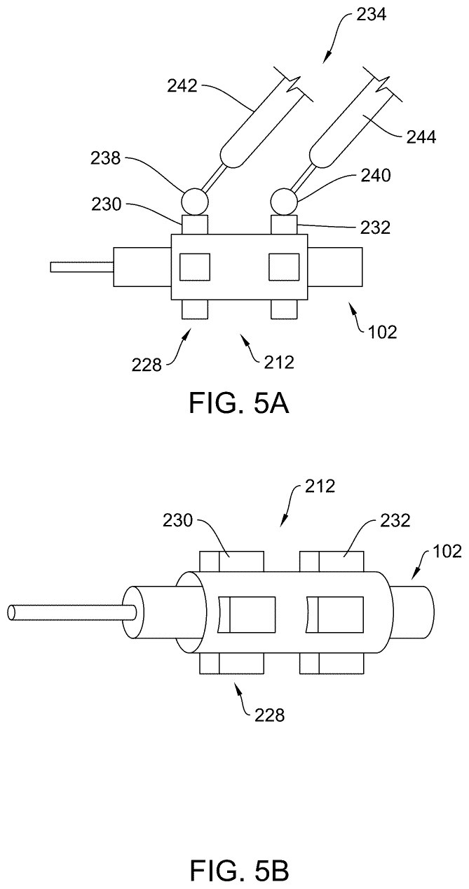

As illustrated in FIGS. 2B and 2D, the clamping body 212 further includes an outer portion 226 opposite of the inner portion 214. The outer portion 226 may include at least one clamping magnet 228. The at least one clamping magnet 228 may be an electromagnet. In some embodiments, the at least one clamping magnet 228 can include two of the clamping magnet 228. For example, as illustrated in FIGS. 2A, 4B, 5A, and 5B, the at least one clamping magnet 228 can include a first clamping magnet 230 and a second clamping magnet 232 adjacent to the first clamping magnet 230. In other embodiments, the at least one clamping magnet 228 can include less than two (e.g., one) or greater than two (e.g., three, five, etc.) of the clamping magnet 228.

As illustrated in FIG. 2A, the positioning portion 210 includes a receiving member 234. The receiving member 234 is configured to couple to the clamping body 212. The receiving member 234 may include at least one receiving magnet 236. The at least one receiving magnet 236 is configured to magnetically couple to the at least one clamping magnet 228 of the clamping body 212. The at least one receiving magnet 236 may be an electromagnet.

In some embodiments, the at least one receiving magnet 236 can include two of the receiving magnet 236. For example, as illustrated in FIGS. 2A and 3B, the at least one receiving magnet 236 can include a first receiving magnet 238 and a second receiving magnet 240 adjacent to the first receiving magnet 238. In other embodiments, the at least one receiving magnet 236 can include less than two (e.g., one) or greater than two (e.g., three, five, etc.) of the receiving magnet 236.

In some embodiments, as illustrated in FIGS. 2A, 4B, and 5A, the first receiving magnet 238 is configured to magnetically couple to the first clamping magnet 230 and the second receiving magnet 240 is configured to magnetically couple to the second clamping magnet 232. In other embodiments, the first receiving magnet 238 is configured to magnetically couple to the second clamping magnet 232 and the second receiving magnet 240 is configured to magnetically couple to the first clamping magnet 230.

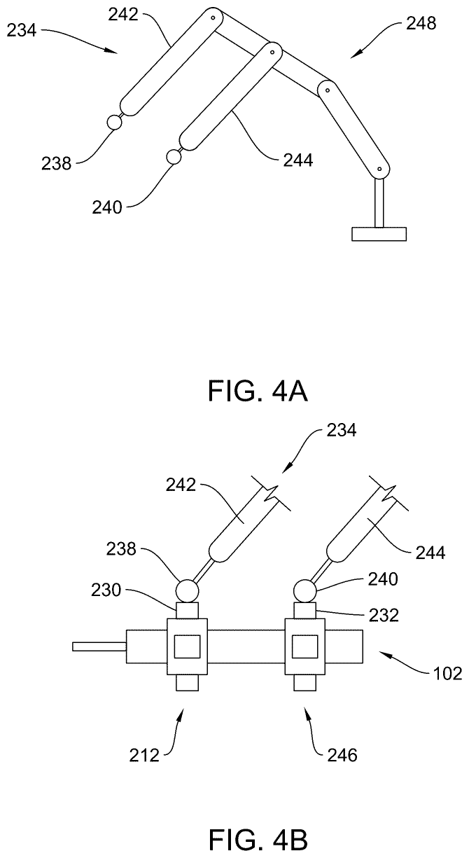

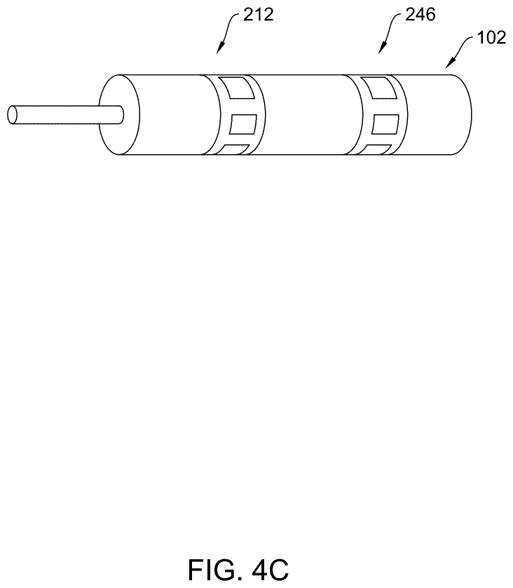

As illustrated in FIGS. 4A and 4B, the receiving member 234 may include a first arm 242 coupled to the first receiving magnet 238 and a second arm 244 coupled to the second receiving magnet 240. In some embodiments, at least one of the first arm 242 or the second arm 244 is telescopic.

In some embodiments, as illustrated in FIGS. 4B and 4C, the clamping body 212 is a first clamping body and the at least one clamping magnet 228 of the first clamping body includes a first clamping magnet (e.g., the first clamping magnet 230). Further in these embodiments, the positioning portion 210 includes a second clamping body 246 that is configured to couple to the medical imaging device 102. The second clamping body 246 may be similar to the first clamping body (e.g., the clamping body 212), such that the disclosure herein with respect to the clamping body 212 applies to the second clamping body 246 unless noted otherwise. The second clamping body 246 can include a second clamping magnet (e.g., the second clamping magnet 232). In some embodiments, the second clamping magnet 232 of the second clamping body 246 is configured to magnetically couple to the second receiving magnet 240. In other embodiments, the second clamping magnet 232 of the second clamping body 246 is configured to magnetically couple to the first receiving magnet 238.

As illustrated in FIG. 4A, the second apparatus 200 further includes a support portion 248. The support portion 248 is coupled to the positioning portion 210 and configured to support the positioning portion 210. The support portion 248 of the second apparatus 200 may be similar to the support portion 120 of the first apparatus 100, such that the disclosure herein with respect to the support portion 120 of the first apparatus 100 applies to the support portion 248 of the second apparatus 200, unless noted otherwise.

A system may include the second apparatus 200 and/or the receiving member 234. As illustrated in FIG. 3B, the system may include a switch 250. The switch 250 may be hand-operated switch, such as a lever, or a foot-operated switch, such as a foot pedal. The switch 250 may be operatable between a first position and a second position. The system may include a controller 252. The controller 252 may be communicatively coupled to at least one of the switch 250, the at least one clamping magnet 228, or the at least one receiving magnet 236. In some embodiments, the controller 252 is communicatively coupled to the switch 250, the at least one clamping magnet 228, and the at least one receiving magnet 236.

The controller 252 may be configured to, when the switch 250 is in the first position, provide electrical current to at least one of the at least one clamping magnet 228 or the at least one receiving magnet 236 to generate magnetic fields. The controller 252 may be configured to, when the switch is in the second position, not provide the electrical current to the at least one of the at least one clamping magnet 228 or the at least one receiving magnet 236 to prevent or minimize generation of the magnetic fields.

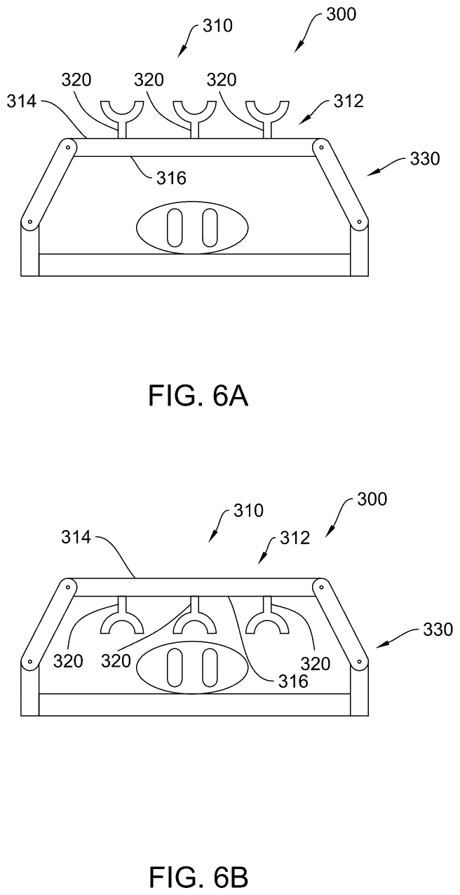

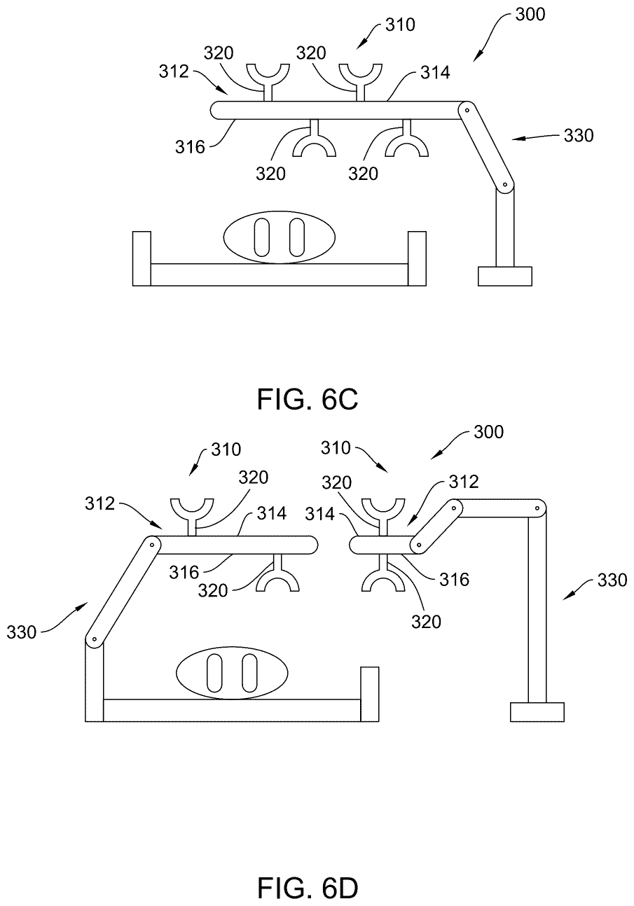

FIGS. 6A-6D illustrate a third apparatus 300 (e.g., an apparatus, etc.) for positioning the medical imaging device 102 during the medical procedure, according to various example embodiments. The third apparatus 300 may position a plurality of medical imaging devices that include the medical imaging device 102. While the third apparatus 300 of FIGS. 6A-6D are shown for holding a plurality of medical imaging devices, it is understood that the third apparatus 300 may, in certain embodiments, be configured to only receive one medical imaging device.

The third apparatus 300 includes a positioning portion 310. The positioning portion 310 is configured to position at least one of the plurality of medical imaging devices during the medical procedure. The positioning portion 310 includes a base 312. The base 312 includes an upper portion 314 and a lower portion 316 opposite of the upper portion 314.

The positioning portion 310 includes a plurality of clips 320. In some embodiments, at least one of the plurality of clips 320 includes a C-clip. The plurality of clips 320 are coupled to the base 312. At least one of the plurality of clips 320 may be biasedly closed. At least one of the plurality of clips 320 may be configured to receive a respective medical imaging device (e.g., the medical imaging device 102) of the plurality of medical imaging devices, grasp at least a portion of the respective medical imaging device of the plurality of medical imaging devices, and substantially spatially fix about a rotational axis (e.g., the rotational axis 104) the respective medical imaging device of the plurality of medical imaging devices. In some embodiments, the positioning portion 310 may have multiple plurality of clips 320 with various sizes and/or shapes to accommodate various medical imaging devices and/or other medical devices.

As illustrated in FIG. 6A, the plurality of clips 320 may be coupled to the upper portion 314 of the base 312. As illustrated in FIG. 6B, the plurality of clips 320 may be coupled to the lower portion 316 of the base 312. As illustrated in FIGS. 6C and 6D, a first portion of the plurality of clips 320 may be coupled to the upper portion 314 of the base 312 and a second portion of the plurality of clips 320 may be coupled to the lower portion 316 of the base 312.

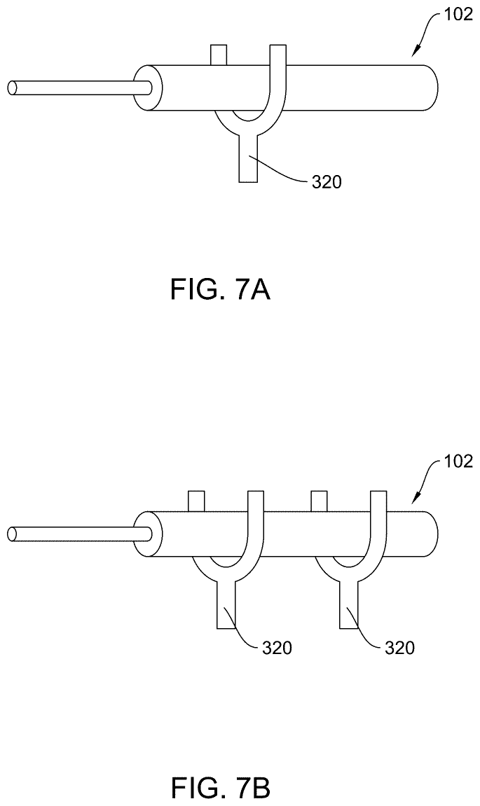

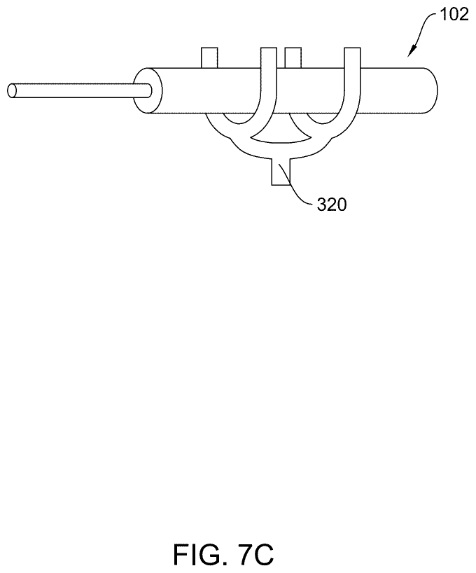

As illustrated in FIG. 7A, the medical imaging device 102 may be configured to be received by one of the plurality of clips 320. As illustrated in FIG. 7B, the medical imaging device 102 may be configured to be received by two of the plurality of clips 320. As illustrated in FIG. 7C, the medical imaging device 102 may be configured to be received by one of the plurality of clips 320 that includes two adjacent C-clips.

As illustrated in FIGS. 6A-6D, the third apparatus 300 may further include a support portion 330 coupled to the positioning portion 310. The support portion 330 is configured to support the positioning portion 310. The support portion 330 of the third apparatus 300 may be similar to the support portion 120 of the first apparatus 100, such that the disclosure herein with respect to the support portion 120 of the first apparatus 100 applies to the support portion 330 of the third apparatus 300, unless noted otherwise.

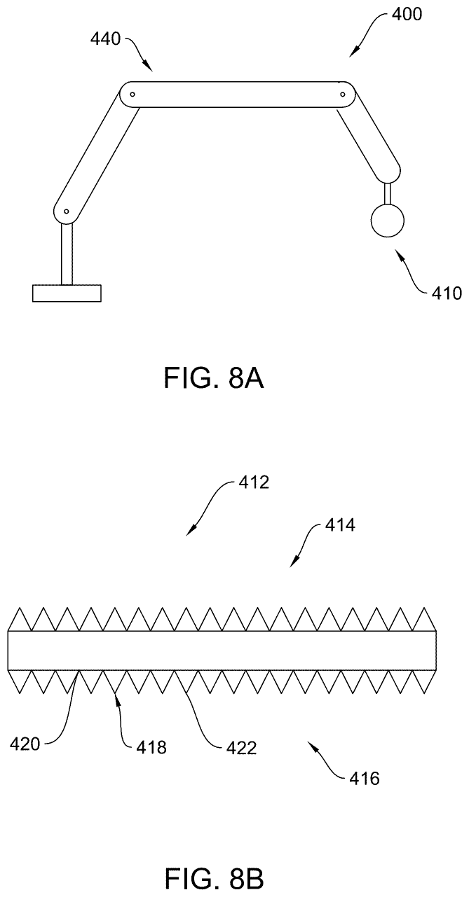

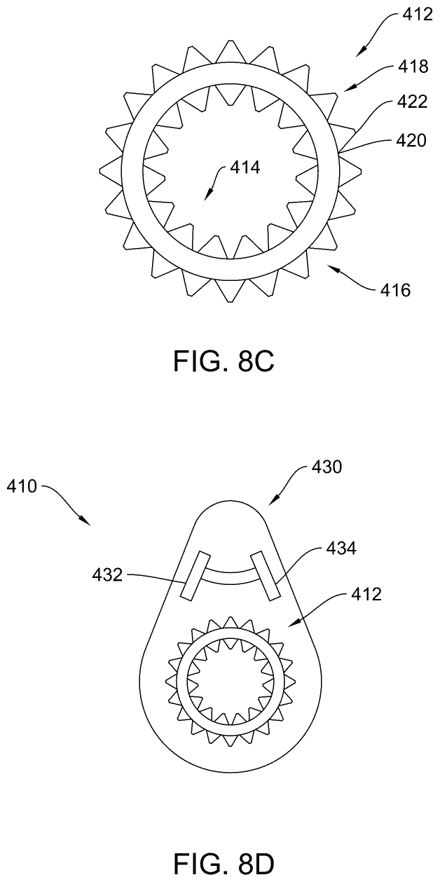

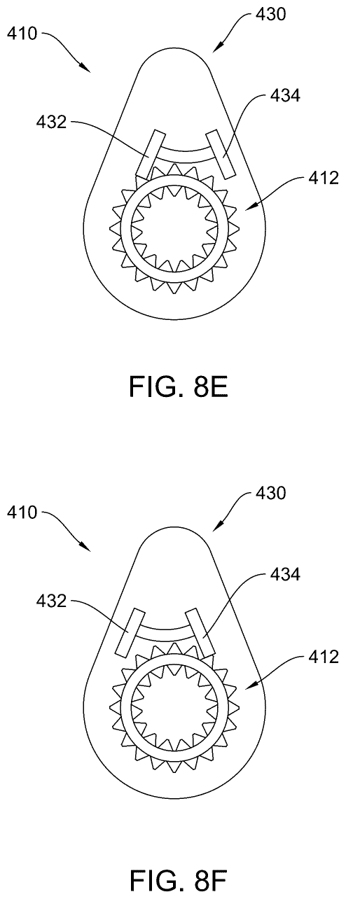

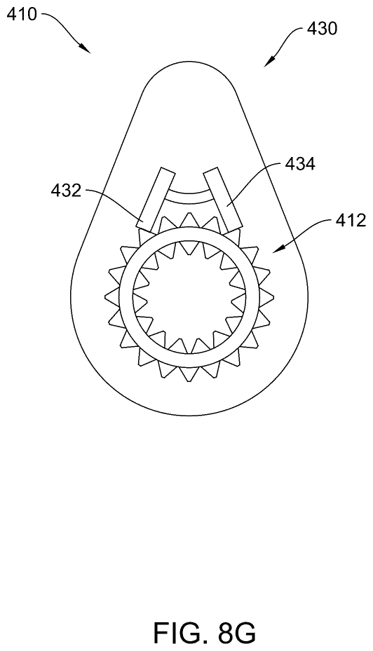

FIG. 8A illustrates a fourth apparatus 400 (e.g., an apparatus, etc.) for positioning the medical imaging device 102 during a medical procedure, according to an example embodiment. The fourth apparatus 400 includes a positioning portion 410. The positioning portion 410 is configured to position the medical imaging device 102 during the medical procedure. The positioning portion 410 includes a clamping body 412. The clamping body 412 is configured to couple to the medical imaging device 102.

As illustrated in FIGS. 8B and 8C, the clamping body 412 includes an inner portion 414 and an outer portion 416. The inner portion 414 is configured to contact the medical imaging device 102. The inner portion 414 of the fourth apparatus 400 may be similar to the inner portion 214 of the second apparatus 200, such that the disclosure herein with respect to the inner portion 214 of the second apparatus 200 applies to the inner portion 414 of the fourth apparatus 400, unless noted otherwise. For example, the inner portion 414 of the fourth apparatus 400 may include a plurality of teeth similar to the plurality of teeth 216 of the inner portion 214 of the second apparatus 200.

The clamping body 412 further includes the outer portion 416 opposite of the inner portion 414. The outer portion 416 includes a plurality of clamping teeth 418 such that the plurality of clamping teeth 418 may be spaced directly adjacent to one another or spaced some distance apart. In some embodiments, the plurality of clamping teeth 418 may be spaced non-uniformly and/or uniformly from one another. The plurality of clamping teeth 418 include a plurality of clamping bottom lands 420. The plurality of clamping bottom lands 420, in some implementations, may be disposed between adjacent teeth of the plurality of clamping teeth 418. In implementations in which the plurality of clamping teeth 418 are spaced apart from one another, the plurality of clamping bottom lands 420 may be defined as the space or portion spanning between adjacent teeth in the plurality of clamping teeth 418. One or more of the plurality of clamping teeth includes a clamping top land 422.

As illustrated in FIGS. 8D-8G, the positioning portion 410 further includes a receiving housing 430. The receiving housing 430 is configured to couple to the clamping body 412. In some embodiments, the receiving housing 430 is configured to receive at least a portion of the clamping body 412. The receiving housing 430 may include a clockwise locking tooth 432. The clockwise locking tooth 432 is configured to interface with at least one of the plurality of clamping bottom lands 420 or at least one of the plurality of clamping teeth 418 to prevent rotation of the clamping body 412 in a clockwise direction. The receiving housing 430 may include a counterclockwise locking tooth 434. The counterclockwise locking tooth 434 is configured to interface with the at least one of the plurality of clamping bottom lands 420 or the at least one of the plurality of clamping teeth 418 to prevent rotation of the clamping body 412 in a counterclockwise direction.

The receiving housing 430 may be operable between a plurality of positions. The plurality of positions may include a first position. As illustrated in FIG. 8D, in the first position, neither of the clockwise locking tooth 432 nor the counterclockwise locking tooth 434 are engaged with the clamping body 412 (e.g., not in contact with the at least one of the plurality of clamping bottom lands 420 or the at least one of the plurality of clamping teeth 418, etc.). In the first position, the clamping body 412 is free to rotate in the clockwise direction and the counterclockwise direction.

The plurality of positions may include a second position. As illustrated in FIG. 8E, in the second position, the clockwise locking tooth 432 is engaged with the clamping body 412 (e.g., in contact with the at least one of the plurality of clamping bottom lands 420 or the at least one of the plurality of clamping teeth 418, etc.) and the counterclockwise locking tooth 434 is not engaged with the clamping body 412. In the second position, the clamping body 412 is prevented from rotating in the clockwise direction and free to rotate in the counterclockwise direction.

The plurality of positions may include a third position. As illustrated in FIG. 8F, in the third position, the counterclockwise locking tooth 434 is engaged with the clamping body 412 and the clockwise locking tooth 432 is not engaged with the clamping body 412. In the third position, the clamping body 412 is prevented from rotating in the counterclockwise direction and free to rotate in the clockwise direction.

The plurality of positions may include a fourth position. As illustrated in FIG. 8G, in the fourth position, both of the clockwise locking tooth 432 and the counterclockwise locking tooth 434 are engaged with the clamping body 412. In the fourth position, the clamping body 412 is prevented from rotating in both the clockwise direction and the counterclockwise direction.

As illustrated in FIG. 8A, the fourth apparatus 400 includes a support portion 440. The support portion 440 is coupled to the positioning portion 410. The support portion 440 is configured to support the positioning portion 410. The support portion 440 of the fourth apparatus 400 may be similar to the support portion 120 of the first apparatus 100, such that the disclosure herein with respect to the support portion 120 of the first apparatus 100 applies to the support portion 440 of the fourth apparatus 400, unless noted otherwise.

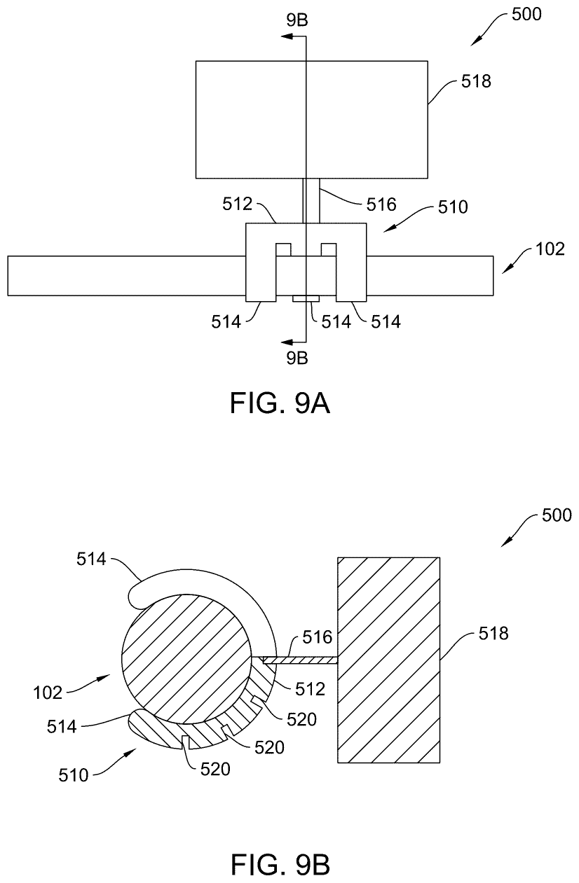

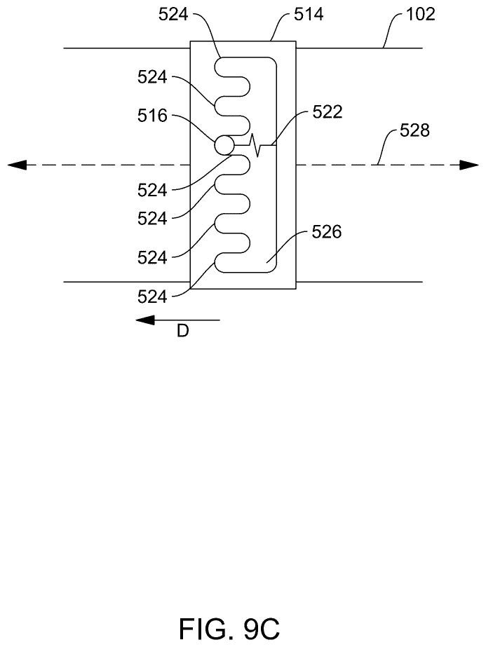

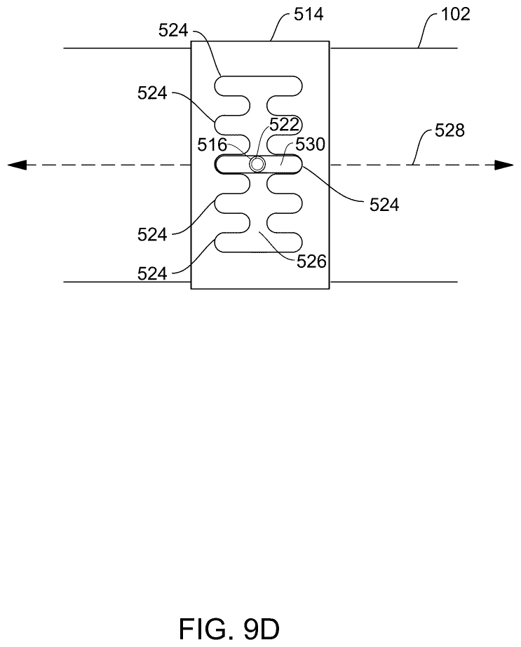

FIGS. 9A and 9B illustrate a fifth apparatus 500 (e.g., an apparatus, etc.) for positioning a medical imaging device 102 during a medical procedure, according to an example embodiment. The fifth apparatus 500 includes a positioning portion 510. The positioning portion 510 is configured to position the medical imaging device 102 during the medical procedure. As illustrated in FIGS. 9A and 9B, the positioning portion 510 includes a base 512 and at least one member 514 coupled to the base 512. The at least one member 514 may be bendable (e.g., flexible, semi-flexible, etc.). The at least one member 514 is configured to grasp at least a portion of the medical imaging device 102 and substantially rotationally fix the medical imaging device 102 about a rotational axis (e.g., the rotational axis 104 of FIG. 1C, etc.).

In some embodiments, the positioning portion 510 includes three of the member 514 (e.g., the at least one member 514 includes three members 514, etc.). In other embodiments, the positioning portion 510 includes less than three (e.g., two, one, etc.) or greater than three (e.g., four, seven, etc.) of the member 514.

In some embodiments, at least one member 514 may include at least one grippable region (e.g., the grippable region 116 of FIG. 1B, a sticky region, a rubber area, etc.). The at least one grippable region is configured to contact the medical imaging device 102 and prevent the medical imaging device 102 from substantially rotating about the rotational axis. In some embodiments, the at least one member 514 includes three of the at least one grippable region. In other embodiments, the at least one member 514 includes less than three (e.g., two, one, etc.) or greater than three (e.g., four, seven, etc.) of the grippable region.

As illustrated in FIGS. 9A and 9B, the fifth apparatus 500 further includes a support portion 516 and a weight 518 (e.g., a weighted object, an object with substantial weight, etc.) coupled to the support portion 516 (e.g., threadedly coupled, adhesively coupled, frictionally coupled, fixedly coupled). The support portion 516, in certain embodiments, is selectively coupled to the positioning portion 510. More specifically, the positioning portion 510 can define a plurality of slots 520. Each of the plurality of positions or slots 520 is configured to selectively receive the support portion 516, thereby selectively coupling the support portion 516 and the weight 518 to the positioning portion 510 such that the positioning portion 510 is rotationally stabilized or fixed via the weight 518. The positioning portion 510 may define the slots 520 within the base 512 and/or the at least one member 514.

The weight 518 may be configured to be placed on a side table, an operating table, the patient, a fixed object, etc. Operations of the fifth apparatus 500 may include coupling the positioning portion 510 to the weight 518 by inserting the support portion 516 within a first of the slots 520, decoupling or repositioning the positioning portion 510 relative to the weight 518 by removing the support portion 516 from the first of the slots 520, rotating the positioning portion 510, and recoupling the positioning portion 510 to the weight 518 by inserting the support portion 516 within a second of the slots 520. In some embodiments, the support portion 516 may be threadedly coupled to the weight 518. In other embodiments, the support portion 516 may be fixedly coupled to the weight 518.

Though not shown, the weight 518 may include one or more contoured side profiles to improve gripping of the weight 518 during angular adjustment of the positioning portion 510 to facilitate operation of the positioning portion 510 (e.g., with one hand, two hands, etc). By way of example, the weight 518 may have one or more indentations on a face of the weight 518 opposite the support portion 516, wherein the one or more indentations may be configured to index one or more fingers of an operator of the fifth apparatus 500 (e.g., the one or more indentations may be in the general shape of a finger to receive one or more fingers of the operator). Likewise, the at least one member 514 may include one or more tabs, flanges, and/or profiles to facilitate one-handed operation of the fifth apparatus 500. For example, the at least one member 514 may include a tab extending therefrom to interact with an operator's finger (e.g., a thumb) during one-handed rotation of the at least one member 514 relative to the weight 518.

The support portion 516 may be telescopic (e.g., extendable, retractable, etc.). In some embodiments, at least one of the slots 520 includes a grippable region therein, such that the support portion 516 is securely coupled to the positioning portion 510 when received within the slot 520. In other embodiments, at least one of the slots 520 includes a magnet therein and the support portion 516 includes a magnetic metal (e.g., a ferrous material), such that the support portion 516 is securely (e.g., magnetically) coupled to the positioning portion 510 when received within the slot 520.

Turning now to FIG. 9C, an additional and/or alternative embodiment of the fifth apparatus 500 is shown. FIG. 9C depicts the medical imaging device 102 received by one of the at least one member 514 of the positioning portion 510, and further illustrates a retention system configured to facilitate discrete locking positions of the support portion 516 along an arc radially positioned, at least in part, about a rotational axis 528 that extends along a longitudinal axis of the medical imaging device 102.

The support portion 516 is shown received within a slot 524 of the member 514. In some embodiments, the slot 524 may be substantially similar to the slots 520 of FIGS. 9A and 9B. A channel 526 defines a curved guide path oriented along the path of rotation of the positioning portion 510 relative to the weight 518. The slots 524 are discretely defined along the channel 526. Each slot 524 may be recessed or undercut relative to the contour of the channel 526 to facilitate engagement with the support portion 516 which may include a head at a distal or proximal region of the support portion 516, and may be spaced apart to correspond with predetermined angular positions of the positioning portion 510 relative to the weight 518 (e.g., 1°, 5°, etc.)

The support portion 516 may include a spring-loaded mechanism for selectively biasing the support portion 516 in a direction D to bias the support portion 516 within the slot 524. A spring 522 may be constrained within the at least one member 514 such that it biases the support portion 516 into the slot 524. The support portion 516 is configured to selectively engage with one of the discrete slots 524 to secure the support portion 516 in a fixed angular position relative to the channel 526. The engagement of the support portion 516 with the slot 524 prevents further rotation of the support portion 516 about the rotational axis 528 until the support portion 516 is retracted into the channel 526 (e.g., by compressing the spring 522) for further positional/angular adjustment. While the at least one member 514 is shown with multiple slots 524 for discrete positioning, it is understood that while not expressly shown in FIG. 9C, in other embodiments the at least one member 514 may have a channel 526 without slots 524, allowing the spring 522 to bias the support portion 516 against a side wall of the channel 526 in any position along the channel 526, thereby providing an infinitely adjustable positioning of the at least one member 514 relative to the weight 518. In other embodiments, the support portion is fixedly coupled to the at least one member 514, thereby resulting in a fifth apparatus 500 that is non-rotationally adjustable. In other embodiments,

In some embodiments, the support portion 516 may be manually retractable (e.g., with one hand) by a clinician to facilitate repositioning of the support portion 516. Upon realignment, the support portion 516 may automatically engage a subsequent slot 524 under the spring force of spring 522, thereby locking the orientation of the positioning portion 510. The arrangement permits rapid, convenient, efficient, and/or secure adjustment of the medical imaging device 102 to any of several predetermined and/or desired angular configurations while maintaining rotational stability during the medical procedure.

Turning now to FIG. 9D, an additional or alternative embodiment of the fifth apparatus 500 for positioning the medical imaging device 102 is shown. It should be understood that the at least one member 514 may be angularly adjusted positionally relative to the weight 518 by translating the support portion 516 relative to the at least one member 514 such that the support portion 516 disengages from the slot 524 (as shown in FIG. 9C), however, alternative or additional embodiments may contemplate the support portion 516 moving between one or more slots 524 in one or more alternative methods. For example, as shown in FIG. 9D, the at least one member 514 may have one or more slots 524 on either side of the channel 526. The support portion 516 may include an interlocking member (shown as head 530) or other profiled member fixedly coupled thereto that may interface with one or more of the slots 524 to positionally/angularly lock the at least one member 514 relative to the weight 518. In at least one embodiment, the support portion 516 may be fixedly coupled to (e.g., integral with, fastened to, etc.) the head 530 at a distal or proximal portion or end of the support portion 516. In a default position, the head 530 may be biased by the spring 522 (e.g., provide a biasing force) into the slot 524 so as to fix the at least one member 514 and medical imaging device 102 in an angular position relative to the weight 518.

The head 530, in certain embodiments, may be disengaged from the slot 524 by providing a force axially or otherwise, depending on the implementation, along the support portion 516 (e.g., forcing the medical imaging device 102 toward or away from the weight 518), thus compressing the spring 522, allowing the head 530 to be removed from the slot 524. Once the head 530 is disengaged from the slot 524, the support portion 516 is free to move along the channel 526 to position the head 530 proximate a discrete slot 524. Once the medical imaging device 102 is at a desired angular position relative to the weight 518, the axial force may be removed from the at least one member 514, allowing the spring 522 to expand back to its default configuration, applying a biasing force on the support portion 516 and/or the head 530 to cause the head to engage with the slot 524. It is understood that although the spring 522 is described as expanding to engage the head 530 with the slot 524, the spring 522 may be configured such that it compresses to engage the head 530 with the slot 524.

The configuration illustrated in FIG. 9D enables indexed angular positioning with tactile feedback and discrete locking, improving procedural stability and repeatability during use. While ten slots 524 are shown in the illustrated embodiment, any suitable number of slots 524 (e.g., more or less than ten) may be employed based on desired angular granularity. In other embodiments, the curved channel 526 may be implemented without discrete slots, such as a channel with friction railings on each side, allowing continuous or infinite adjustment. In some embodiments, the spring 522 biases the head 530 against the inner wall of the channel 526 for frictional engagement.

It is to be appreciated that components of the first apparatus 100, the second apparatus 200, the third apparatus 300, the fourth apparatus 400, and the fifth apparatus 500 disclosed herein may be interchanged or combined. For example, the positioning portion 110 of the first apparatus 100 may be coupled to the support portion 248 of the second apparatus 200 (in addition to or alternative to the positioning portion 210 of the second apparatus 200), the support portion 330 of the third apparatus 300 (in addition to or alternative to the positioning portion 310 of the third apparatus 300), the support portion 440 of the fourth apparatus 400 (in addition to or alternative to the positioning portion 410 of the fourth apparatus 400), and/or the support portion 516 of the fifth apparatus 500 (in addition to or alternative to the positioning portion 510 of the fifth apparatus 500).

In some embodiments, at least one of the plurality of clips comprises a C-clip.

In one embodiment, an apparatus for positioning a medical imaging device. The apparatus comprises a positioning portion configured to position the medical imaging device during a medical procedure. The positioning portion comprises a clamping body configured to couple to the medical imaging device. The clamping body comprises an inner portion configured to contact the medical imaging device, and an outer portion opposite of the inner portion. The outer portion comprises a plurality of clamping teeth. The plurality of clamping teeth comprises a plurality of clamping bottom lands. At least one of the plurality of clamping bottom lands is disposed between adjacent teeth of the plurality of clamping teeth. At least one of the plurality of clamping teeth comprises a clamping top land. The positioning portion further comprises a receiving housing configured to couple to the clamping body. The receiving housing comprises a clockwise locking tooth configured to interface with at least one of the plurality of clamping bottom lands of the plurality of clamping teeth to prevent rotation of the clamping body in a clockwise direction. The receiving housing further comprises a counterclockwise locking tooth configured to interface with at least one of the plurality of clamping bottom lands of the plurality of clamping teeth to prevent rotation of the clamping body in a counterclockwise direction. The apparatus further comprises a support portion coupled to the positioning portion and configured to support the positioning portion.

In some embodiments, the receiving housing is operable between (i) a first position in which neither of the clockwise locking tooth nor the counterclockwise locking tooth are engaged with the clamping body, such that the clamping body is free to rotate in the clockwise direction and the counterclockwise direction, (ii) a second position in which the clockwise locking tooth is engaged with the clamping body and the counterclockwise locking tooth is not engaged with the clamping body, such that the clamping body is prevented from rotating in the clockwise direction and free to rotate in the counterclockwise direction, (iii) a third position in which the counterclockwise locking tooth is engaged with the clamping body and the clockwise locking tooth is not engaged with the clamping body, such that the clamping body is prevented from rotating in the counterclockwise direction and free to rotate in the clockwise direction, and (iv) a fourth position in which both of the clockwise locking tooth and the counterclockwise locking tooth are engaged with the clamping body, such that the clamping body is prevented from rotating in both the clockwise direction and the counterclockwise direction.

In some aspects, the techniques described herein relate to a system including: a positioning portion configured to position a medical imaging device during a medical procedure, the positioning portion including: a clamping body configured to couple to the medical imaging device, the clamping body including: an inner portion configured to contact the medical imaging device including a plurality of teeth or friction surface(s), the plurality of teeth including a plurality of bottom lands, at least one of the plurality of bottom lands disposed between adjacent teeth of the plurality of teeth, and an outer portion opposite of the inner portion, the outer portion including at least one clamping magnet; a first end, a second end opposite of the first end, and an aperture proximate the first end, the aperture configured to receive a bottom land of the plurality of bottom lands of the plurality of teeth; and a receiving member configured to couple to the clamping body, the receiving member including at least one receiving magnet configured to magnetically couple to the at least one clamping magnet of the clamping body; and a support portion coupled to the positioning portion and configured to support the positioning portion, wherein the at least one clamping magnet and the at least one receiving magnet are electromagnets; a switch operable between a first position and a second position; and a controller communicatively coupled to the switch, the at least one clamping magnet, and the at least one receiving magnet, the controller configured to: when the switch is in the first position, provide electrical current to at least one of the at least one clamping magnet or the at least one receiving magnet to generate magnetic fields, and when the switch is in the second position, not provide the electrical current to the at least one of the at least one clamping magnet or the at least one receiving magnet to prevent or minimize generation of the magnetic fields.

In some embodiments, the apparatus and methods described herein include a method of positioning a medical imaging device during a medical procedure. The method comprises positioning at least a portion of the medical imaging device proximate at least one member of the apparatus. The method further comprises adjusting the at least one member around at least a portion of the medical imaging device such that the at least one member grasps at least the portion of the medical imaging device and substantially rotationally fixes the medical imaging device about a rotational axis.

The method may further include rotatably positioning the at least one member of the apparatus while the medical imaging device is positionally fixed relative to the at least one member. Rotatably positioning the at least one member may include releasing a spring-actuated rotating mechanism (e.g., as described in FIGS. 9A-9D), rotating the at least one member (e.g., rotating relative to a weighted base of the apparatus) while maintaining the spring-actuated rotating mechanism in the released state, and engaging the spring-actuated rotating mechanism to positionally lock the at least one member in place relative to the rotational axis. In some embodiments, the operator may release the spring-actuated rotating mechanism by depressing the at least one member toward a weighted base (e.g., with one hand, with two hands, etc.) to compress a spring within the spring-actuated mechanism, thereby releasing a locking mechanism within the spring-actuated mechanism (e.g., as shown in FIGS. 9C-9D). In other embodiments, the operator may release the spring-actuated rotating mechanism by pulling the at least one member away from the weighted base to expand a spring within the spring-actuated mechanism. In yet another embodiment, the operator may release the spring-actuated rotating mechanism by translating the at least one member axially along the axis of rotation of the medical imaging device.

A method for positioning a medical imaging device, the method comprising: positioning the medical imaging device proximate a positioning portion of an apparatus; coupling the medical imaging device at least in part to the positioning portion of the apparatus; and adjusting a rotational position of the apparatus. The method for positioning the medical imaging device, wherein adjusting the rotational position of the apparatus comprises: removing the medical imaging device from the positioning portion of the apparatus; rotating the medical imaging device; and recoupling the medical imaging device to the positioning portion of the medical imaging device. The method of positioning the medical imaging device, wherein adjusting the rotational position of the apparatus comprises: applying a force to the positioning portion of the apparatus to overcome a biasing force of the positioning portion of the apparatus to disengage a support portion of the apparatus from a slot; rotating the positioning portion while the medical imaging device is coupled thereto; and releasing the force to the positioning portion to reapply the biasing force to the positioning portion of the apparatus to engage the support portion with the slot. The method for positioning the medical imaging device, wherein adjusting the rotational position of the apparatus comprises: rotating the positioning portion while the medical imaging device is coupled thereto; applying a force to the positioning portion of the apparatus to overcome a biasing force of the positioning portion of the apparatus to disengage a support portion of the apparatus from a slot; rotating a base of the apparatus to rest upon a support surface; and releasing the force to the positioning portion to reapply the biasing force to the positioning portion.

While this specification contains many specific implementation details, these should not be construed as limitations on the scope of what may be claimed but rather as descriptions of features specific to particular implementations. Certain features described in this specification in the context of separate implementations can also be implemented in combination in a single implementation. Conversely, various features described in the context of a single implementation can also be implemented in multiple implementations separately or in any suitable subcombination. Moreover, although features may be described as acting in certain combinations and even initially claimed as such, one or more features from a claimed combination can, in some cases, be excised from the combination, and the claimed combination may be directed to a subcombination or variation of a subcombination.

As utilized herein, the terms “substantially,” “generally,” “approximately,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the appended claims.

The term “coupled” and the like, as used herein, mean the joining of two components directly or indirectly to one another. Such joining may be stationary (e.g., permanent) or moveable (e.g., removable or releasable). Such joining may be achieved with the two components or the two components and any additional intermediate components being integrally formed as a single unitary body with one another, with the two components, or with the two components and any additional intermediate components being attached to one another.

It is important to note that the construction and arrangement of the various systems shown in the various example implementations is illustrative only and not restrictive in character. All changes and modifications that come within the spirit and/or scope of the described implementations are desired to be protected. It should be understood that some features may not be necessary, and implementations lacking the various features may be contemplated as within the scope of the disclosure, the scope being defined by the claims that follow. When the language “a portion” is used, the item can include a portion and/or the entire item unless specifically stated to the contrary.

Also, the term “or” is used, in the context of a list of elements, in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, Z, X and Y, X and Z, Y and Z, or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

What is claimed is:

1. An apparatus for positioning a medical imaging device during a medical procedure, the apparatus comprising:
a positioning portion configured to position the medical imaging device during the medical procedure, the positioning portion comprising:
a base, and
at least one member coupled to the base and being bendable, the at least one member configured to grasp at least a portion of the medical imaging device and substantially rotationally fix the medical imaging device about a rotational axis; and
a support portion coupled to the positioning portion and configured to support the positioning portion.

2. The apparatus of claim 1, wherein the support portion comprises an adjustable arm, the adjustable arm comprising:
a first member; and
a second member rotatably coupled to the first member.

3. The apparatus of claim 2, wherein the first member and the second member are substantially rigid,
wherein the medical imaging device is a catheter.

4. The apparatus of claim 1, wherein the support portion comprises:
a first end proximate the positioning portion;
a second end opposite the first end; and
a coupling device proximate the second end, the coupling device configured to couple to an object and affix at least a portion of the support portion to the object.

5. The apparatus of claim 4, wherein the coupling device is at least one of a magnet, a clamp, or a screw.

6. The apparatus of claim 4, wherein the coupling device is couplable along a length of the object.

7. The apparatus of claim 1, wherein:
the at least one member comprises three members; and
the support portion comprises a telescopic arm.

8. The apparatus of claim 1, wherein the at least one member comprises at least one grippable region configured to contact the medical imaging device and prevent substantial rotation about the rotational axis, the at least one member is manually disengageable to manually rotate the medical imaging device about the rotational axis.

9. The apparatus of claim 1, wherein the support portion includes:
a first end;
a telescopic arm proximate the first end of the support portion, the telescopic arm rotatably coupled to the base of the positioning portion, the telescopic arm comprising:
a first telescopic end proximate the base of the positioning portion, and
a second telescopic end opposite of the first telescopic end;
an adjustable arm coupled to the telescopic arm proximate the second telescopic end of the telescopic arm, the adjustable arm comprising:
a first member, and
a second member rotatably coupled to the first member; and
a clamp positioned adjacent to the first member of the adjustable arm, the clamp configured to couple to an object and spatially fix the support portion to the object.

10. The apparatus of claim 1, further comprising a weight coupled to the support portion, wherein the positioning portion defines a plurality of slots, at least one of the plurality of slots configured to selectively receive at least a portion of the support portion.

11. The apparatus of claim 10, further comprising a spring mechanism, wherein the spring mechanism is configured to provide a biasing force to the support portion to engage the support portion into one or more of the plurality of slots.

12. An apparatus for positioning a medical device, the apparatus comprising:
a positioning portion configured to position the medical device during a medical procedure, the positioning portion comprising:
a clamping body configured to couple to the medical device, the clamping body comprising:
an inner portion configured to contact the medical device, and
an outer portion opposite of the inner portion, the outer portion comprising at least one clamping magnet; and
a receiving member configured to couple to the clamping body, the receiving member comprising at least one receiving magnet configured to magnetically couple to the at least one clamping magnet of the clamping body; and
a support portion coupled to the positioning portion and configured to support the positioning portion.

13. The apparatus of claim 12, wherein:
the inner portion of the clamping body comprises a plurality of teeth, the plurality of teeth comprising a plurality of bottom lands, at least one of the plurality of bottom lands disposed between adjacent teeth of the plurality of teeth; and
the clamping body further comprises:
a first end,
a second end opposite of the first end, and
an aperture proximate the first end, the aperture configured to receive a bottom land of the plurality of bottom lands of the plurality of teeth.

14. The apparatus of claim 13, wherein at least one tooth of the plurality of teeth comprises rubber.

15. The apparatus of claim 12, wherein:
the at least one clamping magnet of the clamping body of the positioning portion comprises:
a first clamping magnet, and
a second clamping magnet adjacent to the first clamping magnet;
the at least one receiving magnet of the receiving member of the positioning portion comprises:
a first receiving magnet, and
a second receiving magnet;
the first receiving magnet is configured to magnetically couple to the first clamping magnet; and
the second receiving magnet is configured to magnetically couple to the second clamping magnet.

16. The apparatus of claim 15, wherein the receiving member of the positioning portion comprises:
a first arm coupled to the first receiving magnet; and
a second arm coupled to the second receiving magnet.

17. The apparatus of claim 12, wherein:
the clamping body is a first clamping body;
the at least one clamping magnet of the first clamping body comprises a first clamping magnet;
the positioning portion further comprises a second clamping body configured to couple to the medical device, the second clamping body comprises a second clamping magnet;
the receiving member comprises a first arm and a second arm; and
the at least one receiving magnet of the receiving member of the positioning portion comprises:
a first receiving magnet coupled to the first arm, the first receiving magnet configured to magnetically couple to the first clamping magnet of the first clamping body, and
a second receiving magnet coupled to the second arm, the second receiving magnet configured to magnetically couple to the second clamping magnet of the second clamping body.

18. An apparatus for positioning a catheter, the apparatus comprising:
a positioning portion configured to position the catheter during a medical procedure, the positioning portion comprising:
a base comprising:
an upper portion, and
a lower portion opposite the upper portion; and
one or more clips coupled to the base, at least one of the one or more clips configured to receive the catheter, grasp at least a portion of the catheter, and substantially spatially fix the catheter about a rotational axis of the catheter; and
a support portion coupled to the positioning portion and configured to support the positioning portion.

19. The apparatus of claim 18, wherein the one or more clips are coupled to the upper portion of the base of the positioning portion and are configured to position a plurality of catheters.

20. The apparatus of claim 18, wherein the one or more clips are coupled to the lower portion of the base of the positioning portion and are configured to position a plurality of catheters.
